(19) United States
(12) Patent Application Publication
(10) Pub. No.: US 2020/0327972 A1
(43) Pub. Date: Oct. 15, 2020

(54) AUTOMATIC DETECTION OF MEDICAL ADHERENCE

(71) Applicants: Arunprasad Ramiya Mothilal, Pleasanton, CA (US); Tapan Samaddar, Sunnyvale, CA (US)

(72) Inventors: Arunprasad Ramiya Mothilal, Pleasanton, CA (US); Tapan Samaddar, Sunnyvale, CA (US)

(21) Appl. No.: 16/379,772

(22) Filed: Apr. 9, 2019

(51) Int. Cl. G16H 20/13; G01P 13/00

(52) U.S. Cl. CPC G16H 20/13; G01P 13/00

(57) ABSTRACT

A system and a process for automatic detection of medical adherence in a medical device is disclosed. The process includes automatically detecting a state change of a medical device based on a motion of the medical device. The state change may be from a closed state to an open state. It is determined whether a state change processor is within a range of the medical device. The state change processor monitors medical or medicine adherence. Upon determining that the state change processor is within the range of the medical device, state change information is sent to the state change processor. A real time change data when the medical activity detector and the state change detector are within a communication range is stored.

### TECHNICAL FIELD

The invention is generally related to the field of medical adherence and more particularly related to the field of medical devices and the state of medical device.

### BACKGROUND

In medicine, compliance also referred to as adherence is the extent to which a patient follows medical advice. It is also referred to as medication or drug compliance in some cases. When the patient fails to comply or adhere to the medical advice it results in non-compliance. In United States, every five minutes a person dies because of medication non-adherence. Various solutions are available in the market to improve medication adherence. Most solutions include a compact portable medicine container with battery, LED, buzzer to remind the users and a display to indicate when the last dose of medication was taken. This solution may be easy to use, but does not allow the users to schedule notification or notify caretaker or health care personnel. The medical adherence solutions available in the market at this point of time are expensive, very complex to setup, some of them not portable and does not notify caretaker and not practical for everyday use. It is challenging to have a medical device that is easy to use, compact and portable, not very expensive, and flexible to add various software functionalities.

### BRIEF DESCRIPTION OF THE DRAWINGS

The claims set forth the embodiments with particularity. The embodiments are illustrated by way of examples and not by way of limitation in the figures of the accompanying drawings in which like references indicate similar elements. Various embodiments, together with their advantages, may be best understood from the following detailed description taken in conjunction with the accompanying drawings.

FIG. 1A to FIG. 1F is a block diagram illustrating high level architecture of a system to detect a medical adherence, according to one embodiment.

FIG. 2 illustrates a medical device along with a medicine container in a closed position, according to one embodiment.

FIG. 3 illustrates a medical device along with a medicine container in a closed position, according to one embodiment.

FIG. 4 illustrates a medical device along with a medicine container in an open position, according to one embodiment

FIG. 5 is a diagram illustrating a container cap in an open position, according to one embodiment.

FIG. 6 illustrates an exploded view of medical device, according to an embodiment.

FIG. 7 illustrates an exploded view of medical device, according to one embodiment.

FIG. 8 is a block diagram illustrating medical activity detector in a printed circuit board assembly (PCBA), according to one embodiment.

FIG. 9 illustrates a block diagram of top view of an electronic circuit including the medical device, according to one embodiment.

FIG. 10 illustrates a block diagram of bottom view of an electronic circuit including the medical device, according to one embodiment.

FIG. 11 is a flow diagram illustrating a process of event detection, according to an embodiment.

FIG. 12 is a flow diagram illustrating a process of communication of a medical device with an application software, according to an embodiment.

FIG. 13 is a flow diagram illustrating a process for automatic detection of medical adherence in a medical device, according to an embodiment

FIG. 14 is a block diagram illustrating a computing system consistent with implementations of the current subject matter.

Although the specific features of the present invention are shown in some drawings and not in others. This is done for convenience only as each feature may be combined with any or all of the other features in accordance with the present invention.

### DETAILED DESCRIPTION OF THE INVENTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense.

In the following detailed description, it may be noted that “medical adherence” refers to “medicine adherence.” However, in various embodiments, it may not only be restricted to “medicine adherence” and may cover its equivalent(s) thereof as well.

The various embodiments disclose a system and method to automatically detect medical adherence. In one embodiment, the medical adherence is a process of detecting the degree or extent of patient's compliance to a medical advice. The medical advice may be received through an app (application software with an user interface) in a patient's computing device. The computing device (state change processor) may be a smartphone, smart tablet, or a laptop or personal computer or any portable electronic device or gadget. Medical device may include a housing top, a battery, a battery holder, a medical activity detector that is a printed circuit board, a switch, a nylon washer, a water proof lid, a medicine bottle cap housing bottom, and a medicine container bottle head. A medical device allows a smartphone app to connect and enable monitoring medical adherence of a patient. If the medical device is within the range of the smartphone app, the medical device may communicate the medical advice such as medicine taken/to be taken notifications to the smartphone app. The smartphone app determines whether the patient has taken the prescribed medication or not. If the medical device is not in the smartphone range, then the medical device records the medicine taken information and communicates to the smartphone app when the smartphone app is within the range of the medical device. The smartphone app may be a proprietary software application capable of ensuring medical adherence of patients via the medical device. The medical device and the smartphone app work in unison and ensures medical adherence of patients. Medical adherence includes auto monitoring the patient's consumption of correct medicine in correct dosage of the medicine at a prescribed time. For example, a doctor may have prescribed 5 ml of medicine A to be consumed twice a day in the morning and the evening with a time interval of 6 hours between the two doses. In this scenario, the medical device along with the smart phone App will check whether the patient has consumed the medicine between 7-9 am in morning and 5-7 pm in evening and whether the time interval of 6 hours was adhered between these two dosages.

In one embodiment, a caretaker or user may detect the medical adherence by using a medical device. The medical device is in the form of a cap that includes a medical activity detector. The medical device fits on top of the medicine container as a cap. The medical activity detector detects any change in the state of the medical device. The change in the state can be from an open state to a closed state, or from the closed state to the open state. The state change such as open and close may take place by a press mechanism where for example, the press mechanism is performed on an eye drop bottle. The state change such as open and close may take place by a push mechanism where for example, the push mechanism is used to push an insulin pen. The change in state can be change in the medicine amount inside the medicine container. Further, change in the medicine or empty state of the medicine in the medicine container may be notified by the activity detector in the medical device. For example, the state of the medical device may be “open” where the medical device is open and the medicines can be dispensed or poured out of the medicine container. The state of the medical device may be “closed” where the medical device is closed and medicines cannot be dispensed or poured out of the medicine container. A change in state of the medical device in this scenario may be from an “open” state to a “close” state, or from the “close” state to the “open” state.

The medical activity detector may detect the change in the state of the medical device based on one or more sensors included in the medical activity detector. A change in state may indicate that a patient has opened the medical device and consumed the medicine. After the medical activity detector detects the change in the state of the medical device, the detected state change is communicated to a “state change” processor that process the state change to detect the patient's medical adherence, i.e., patient taking medicine at the correct prescribed time.

In one embodiment, the “state” change processor may also send a proactive notification to a patient or a caretaker when the “state change” processor does not receive a state change communication from the medical activity detector at the prescribed time. For example, when the time of next medicine dosage is 4 pm and the medical activity detector does not receive a state change message at 4 pm then the medical activity detector indicates that the patient has missed the dosage. In this case, the “state change” processor sends a notification to the patient or a caretaker reminding them of the missed dosage.

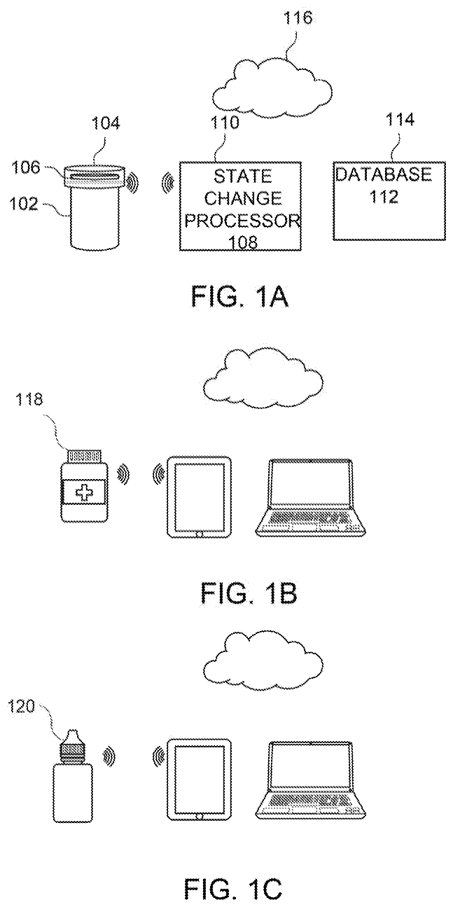

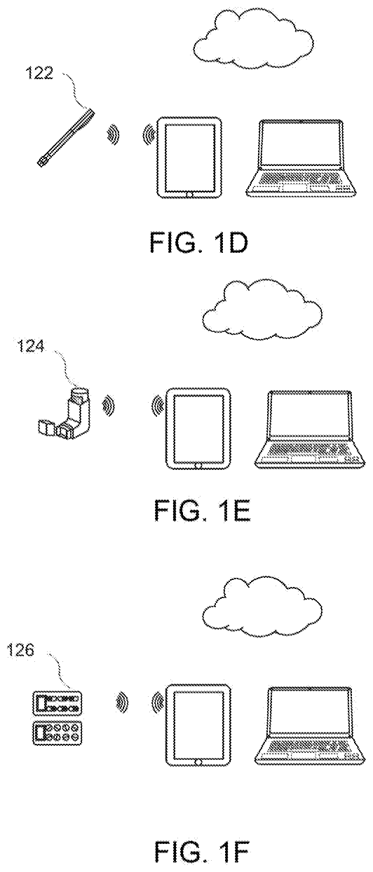

FIG. 1A is a block diagram illustrating high level architecture of system to detect a medical adherence, according to an embodiment. The system includes medicine container 102 with medical device 104 also referred to as a cap, screwed or coupled to the medicine container 102. The medicine container 102 holds medicine in a solid, semi-solid, or liquid form. For example, the medicine container 102 may hold tablets, syrups, eye drops, pre-loaded medicine pens, inhalers, etc. The medicine container 102 has the medical device 104 i.e., the container cap that is used to close the medicine container. The medical device 104 has a lid and medical activity detector 106.

In one embodiment, the lid of the medical device 104 is waterproof to ensure that the medicine is not contaminated by the different components of the medical activity detector 106. The medical activity detector 106 is an ultra-low power smart device with a battery that detects the medicine dispensed from the medicine container 102 by a push and press mechanism or an open/close mechanism or peeling a blister pack. The medical activity detector 106 may include a plurality of sensors to detect a state change of the medical device 104. For example, the medical activity detector 106 may include a snap action switch, a mechanical switch or a tactical switch.

The medical activity detector 106 may also include a low power gyroscope sensor, an accelerometer, a Hall-IC and magnet, a resistor network circuit sensor, a force sensor, a pressure sensor, a temperature sensor, or a combination of one or more of these sensors. The medical activity detector 106 can send the state change of the medical device 104 by the different sensors to a state change processor 108 in wireless device 110 using a wireless communication protocol (WAP).

WAP is a technical standard for accessing information over a mobile wireless network. The wireless protocols belong to three main range of classes such as long range, medium range and short range. Long range is measured in miles, medium range is measured in tens or hundreds of feet, while short range is generally less than 100 feet. Each class has different protocols, with varying attributes that make the selection depending on the situation. The medical activity detector 106 can send the state change of the medical device 104 by the different sensors to a state change processor 108 in wireless device 110 using short range and medium range wireless communication protocols. The medical activity detector 106 may include a unique serial number to identify the medical activity detector 106 and the medicine included in the medicine container 102. The medical activity detector 106 may also include a LED, buzzer, vibrator to notify the status of the medical activity detector 106 or a detected activity at the medical device 104.

The state change processor 108 can communicate with the medical activity detector 106 using any encrypted or un-encrypted wireless communication interface and provide an interface to the user to schedule medication, give timely alerts or reminders to take medicine, and notify healthcare personnel or caretakers through email or text message or phone call. In one embodiment, the state change processor 108 may receive the state change message in real-time when the state change processor 108 and the medical activity detector 106 are within the communication range. In case the state change processor 108 and the medical activity detector 106 are not within the communication range, the state change processor 108 receives recorded state change message from the medical activity detector 106. Depending upon the state change data from the medical activity detector 106 at a scheduled time slot, the state change processor 108 could record the medicine taken or skipped event. The state change processor 108 sends the taken or missed medication data to a database 112 in the computer system 114 so that the data can be stored anonymously and analysed to improve medical adherence. In one embodiment, the state change processor 108 also sends the taken or missed medication data to the computer system 114 to access medical adherence data analytics and its reports.

The application that communicates with the medical device 106, wireless device 110 and the computer system 114 such as a smart phone or a smart tablet is hosted in cloud server 116. A cloud server is primarily an Infrastructure as a Service (IaaS) based cloud service model. The cloud server is a logical server that is built, hosted and delivered through a cloud computing platform over the internet. Cloud servers possess and exhibit similar capabilities and functionality to a typical server but are accessed remotely from a cloud service provider. The computer system 114 includes a user interface to display the data analytics and its reports. The analytics is performed in the cloud application and the analytic results are displayed to the user through the computer system 114. The analytics is stored in the database 112.

FIG. 1B to FIG. 1F shows various embodiments of the medical device along with the medicine container in various forms. For example, FIG. 1B shows a medicine bottle 118 holding medicine in the form of a tablet. FIG. 1C shows a medicine container 120 holding medicine in the form of a liquid. FIG. 1D shows the medicine container in the form of an epipen 122 such as insulin pen. FIG. 1E shows the medicine container in the form of an inhaler 124 and the medicine is in the form of aerosol. FIG. 1F shows the medicine container as a blister pack 126

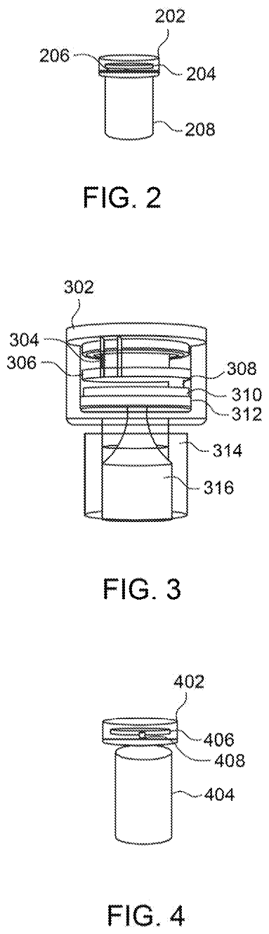

FIG. 2 illustrates a medical device along with a medicine container in a closed position, according to one embodiment. The medical device 202 is shown in the closed position. The medical device 202 includes medical activity detector 204 and waterproof lid 206 inside the medical device 202. The medical device 202 is used as a cap to close the medicine container 208. The medicine container 208 is a container to hold the medicine. For example, the medicine container 102 may hold tablets, syrups, eye drops, pre-loaded medicine pens, inhalers, or medicine blister pack, etc. In closed position, the medical device 202 has the lid shut off where the medicines cannot be dispensed. In the closed position of the medical device 202, the waterproof lid 206 is used to prevent the medicine from being contaminated and the medicine is prevented from coming in contact with the medical device 202. The medicine container 208 may use a thread/screwing mechanism or twist/turn mechanism or flip lid mechanism to close and open the medical device 202. The medical device 202 is placed on the medicine container 208 using any one of the threaded/screwing mechanism, twist and turn mechanism, flip-lid mechanism, etc. Each of these mechanisms provides a different open/close mechanism that is proprietary to the mechanism. The medical device 202 is a compact device that is small in size and easy to use. The medical device 202 may range in a size of few millimetres.

FIG. 3 illustrates a medical device along with a medicine container in a closed position, according to an embodiment. As shown, the medical device includes a housing top 302, a battery holder 304, a medical activity detector that is a printed circuit board 306, a switch 308, a nylon washer 310, a water proof lid 312, a medicine bottle cap housing bottom 314, and a medicine container bottle head 316. Medicine container bottle head 316 pushes the water proof lid 312 and the nylon washer 310 which in-turn closes the switch 308. This closes the medical device so that no medicine from the medicine container is dispensed. Closed position of the medical device locks the device from dispensing medicine.

FIG. 4 illustrates a medical device along with a medicine container in an open position, according to one embodiment. As shown, in the closed position the medical device 402 includes a medicine container 404 to hold the medicine, the medical device 402 to close the container, a medical activity detector 406, and waterproof lid inside the medicine cap 408. In the open position, the medical device 402 opens to dispense medicine from the medicine container 404. In the closed position the medical device 402 is intact and in a closed position over the medicine container 404. When the medical device 402 is in the closed position no medicine is dispensed. The medicine container 404 shown here is merely exemplary, however, the size and shape of the medicine container 404 may vary depending upon the type of medicine used.

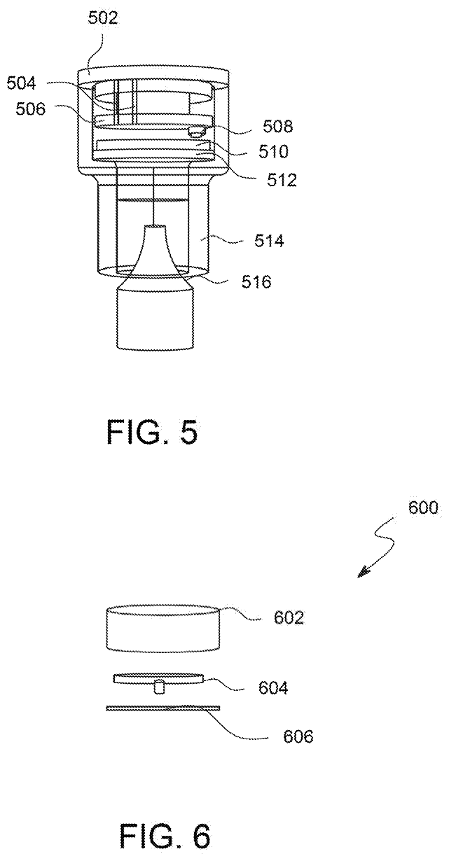

FIG. 5 is a diagram illustrating a container cap in open position, according to an embodiment. As shown the medicine bottle cap has a housing top 502, a battery holder 504, a medical activity detector/printed circuit board 506, a switch 508, a nylon washer 510, a water proof lid 512, a medicine bottle cap housing bottom 514, and a medicine container bottle head 516. Medicine container's head 514 away from the water proof lid 512 and the nylon washer 510 which in-turn opens the switch 508.

FIG. 6 illustrates an exploded view of medical device 600, according to an embodiment. The medical device 600 includes a medicine cap 602, medical activity detector along with the switch 604 and waterproof lid 606. The exploded view shows the parts of the medical device. The waterproof lid 606 ensures that the medicine in the medicine container does not come in contact with the medical activity detector in the medical device 600.

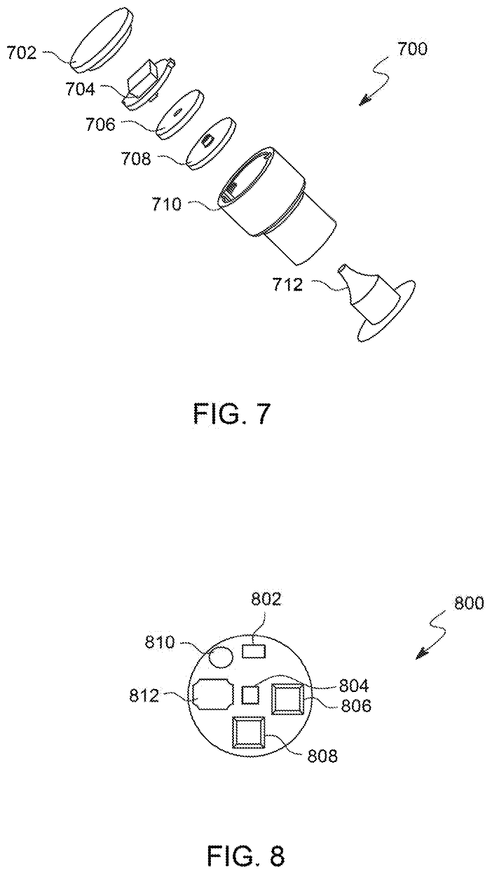

FIG. 7 illustrates an exploded view of medical device 700, according to one embodiment. The medical device 700 includes medicine bottle cap housing top (702), medical activity detector (704), nylon washer (706), silicone or rubber seal (708), medicine bottle cap bottom housing (710), and medicine container bottle head (712). The washer 706 may be made of any durable leak proof material. Similarly, the rubber seal 708 may also be made of any durable material so that the medicine does not leak into the medical device 700. Each of these parts fits into one another forming the complete intact medical device 700. The assembly of these parts is simple for any person skilled in the art to assemble.

FIG. 8 is a block diagram illustrating medical activity detector 800 in a printed circuit board assembly (PCBA), according to one embodiment. The medical activity detector 800 includes an LED 802, switch 804, integrated circuit (IC) 806 that can communicate to the application software and can detect open & close or push/press events of the medical device 800, inertial measurement unit (IMU) 808 that can detect open and close through motion detection, buzzer 810, and battery 812. The LED 802 glows when the medical device is switched ON or if there is a notification to be provided to the user. The LED 802 may be accompanied by a beep or buzzer sound in synchronization to attract the attention of the user/patient. The IMU is an electronic device that measures and reports a body/objects specific force, angular rate and magnetic field surrounding the body, using a combination of accelerometers, gyroscopes and at times magnetometers. The IMU enables detection of the motion of the switch. The battery 812 may be replaced when required ensuring the proper functioning of the medical device. The PCB may include a real time clock (RTC) to store the events with a time stamp.

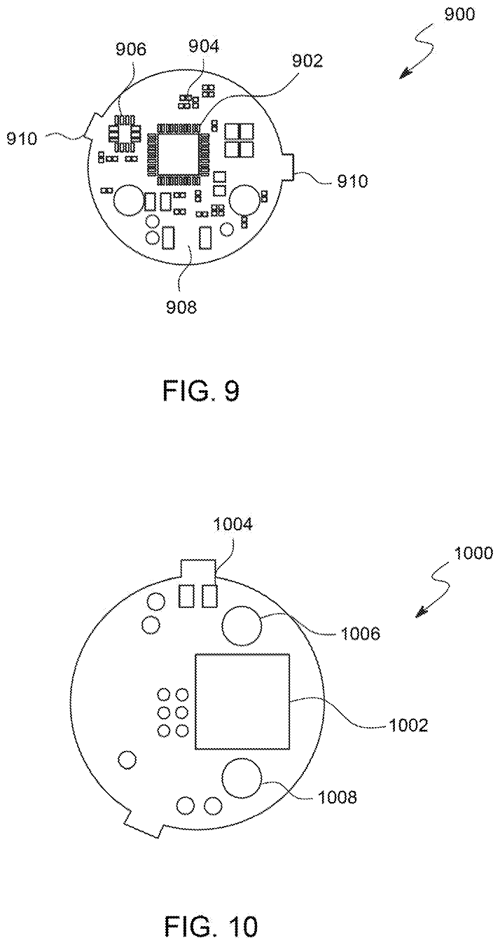

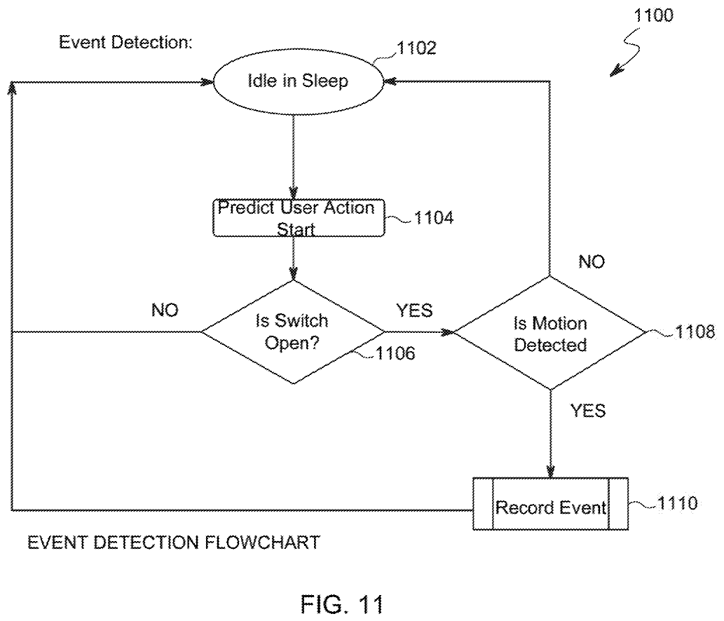

FIG. 9 illustrates a block diagram of top view of an electronic circuit including the medical device 900, according to one embodiment. The electronic circuit includes the Bluetooth, integrated circuit (IC), antenna, switch, sensor, etc. The integrated circuit (IC) 902 is used to communicate with an application software that can detect open and close events associated with a switch in the medical device 900. Radio frequency (RF) antenna 904 is used to transmit radio frequency signal from the medical device 900. The radio frequency signal is an electrical signal emitted as a radio wave. It travels from the broadcasting antenna to the receiving antenna. The RF signal from the medical device 900 is received by a wireless device within the radio frequency range. The inertial measurement unit (IMU) 906 can detect open and close of the switch 908 through motion detection. There is also a protrusion 910 for mechanical alignment with the cap.

FIG. 10 illustrates a block diagram of bottom view of and electronic circuit including the medical device 1000, according to one embodiment. The medical device 900 includes a battery and battery holder 1002, LED 1004 and buzzers 1006 and 1008. The battery holder enables holding of the battery intact in position.

FIG. 1 is a flow diagram illustrating a process 1100 of event detection, according to an embodiment. Initially, at 1102, an activity detector in a medical device is in an idle state referred to as a sleep state. The idle state of the medical device may be interrupted by either an open event or a close event or a change in temperature event, and the medical device specifically the medical activity detector is woken up from the idle state. The open event is determined when a switch is moved from a close state to an open state. Similarly, the close event is determined when the switch is moved from the open state to the close state. Event detection may also be performed when there is a press event or a push event that closes or open the switch. Event detection may also be performed when there is a change in temperature from a pre-programmed range. At 1104, when any one of the open event, close event, press event or the push event takes place, the user action is predicted to have initiated or started. At 1106, it is determined whether the switch is open. Upon determining that the switch is open, at 1108, it is determined whether a motion is detected based on the motion detected through the IMU. Upon determining that the switch is not open, it is inferred that the medical device is in the idle sleep state. If the motion is detected, at 1110, the motion detected is recorded as an event and stored in the internal memory of the medical device. A change in temperature from pre-programmed temperature range is recorded as an event and stored in the internal memory of the device.

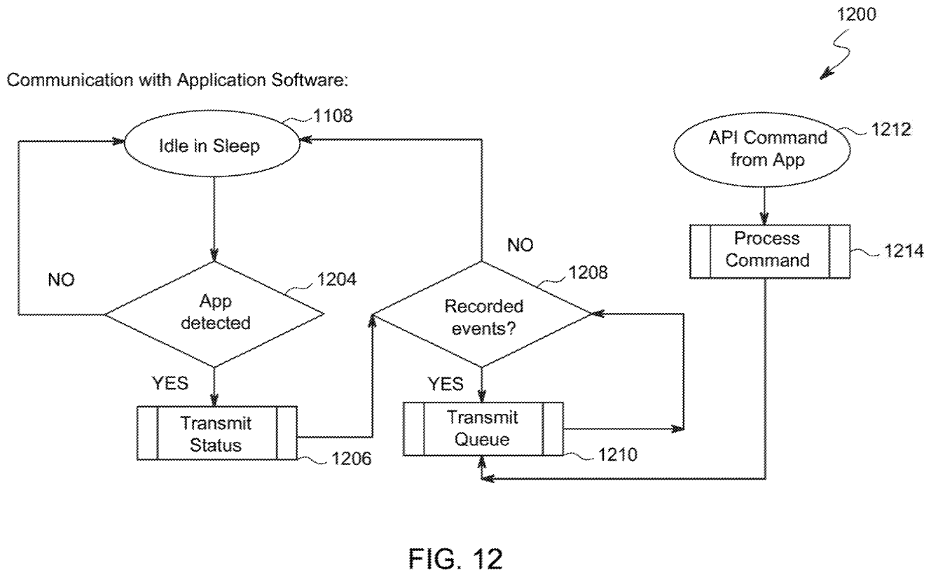

FIG. 12 is a flow diagram illustrating a process 1200 of communication of a medical device with an application software, according to an embodiment. Initially, at 1202, an activity detector in a medical device is in an idle state referred to as a sleep state. At 1204, the idle state of the medical device may be interrupted by detecting an app within a communication range. Upon determining that the app is detected within the communication range, at 1206, the status of the medical device is transmitted to the app. At 1208, it is determined whether the recorded events are available at the medical device. Upon determining that the recorded events are available at the medical device, at 1210, The recorded events are transmitted to the app in a transmit queue, i.e., in the order they were recorded and stored. At 1212, it is determined whether any application programming interface (API) commands are received from the app. At 1214, upon determining that the API commands are received from the app, the commands are processed at the medical device.

The app has the capability to filter out the false positives like opening and closing the container too fast, or not opening the container completely. Such false positives if removed from the event queue provide the patient with accurate results.

There are various advantages of the various embodiments described above. The medical device is easy to use and simple to assemble. The medical device is used to send proactive notification to the patient or caretaker. The medical device is used to track the state change such as open and close of the medical device. The medical activity detector includes a waterproof lid to prevent medicine from coming in contact with the medical activity detector which may be avoid corrosion of the medical activity detector and also contamination of the medicine. Patients are proactively reminded of their medicines on time or at the pre-defined time. This ensures that the patients adhere to their prescribed medicines. The medical device ensures complete medical adherence of the patients. The medical device is inexpensive as well.

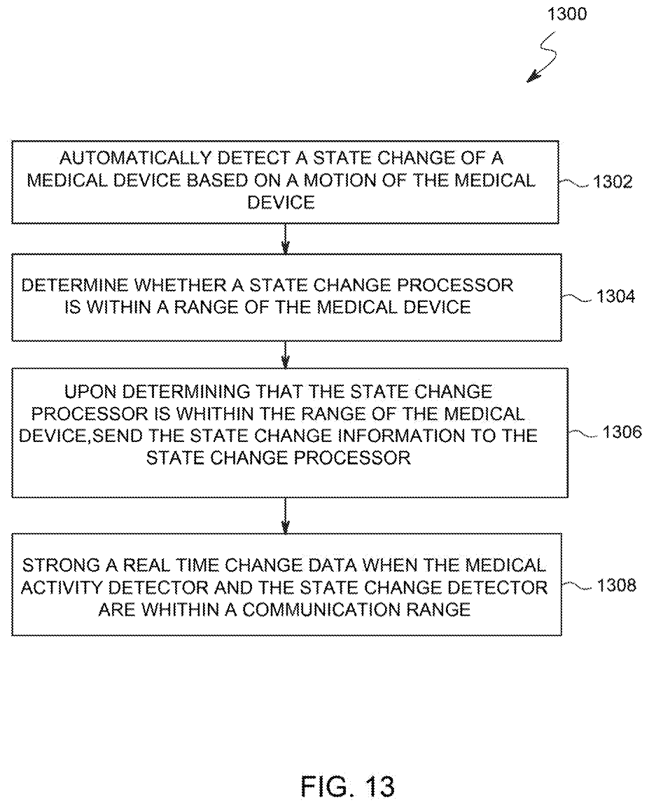

FIG. 13 is a flow diagram illustrating a process 1300 for automatic detection of medical adherence in a medical device, according to an embodiment. At 1302, a state change of a medical device based on a motion of the medical device is automatically detected. The state change is from a first state to a second state. Here, the first state and the second state may be a closed state, open state or vice-versa. At 1304, determine whether a state change processor is within a range of the medical device. The state change processor monitors medical adherence. Upon determining that the state change processor is within the range of the medical device, at 1306, state change information is sent to the state change processor. At 1308, a real time change data when the medical activity detector and the state change detector are within a communication range is stored.

An exemplary embodiment is described below. The “state” change processor may also send a proactive notification to a patient or a caretaker when the “state change” processor does not receive a state change communication from the medical activity detector at the prescribed time. For example, when the scheduled time of medicine dosage is 6 pm and the medical activity detector does not receive a state change message at 6 pm then the medical activity detector indicates that the patient has missed the scheduled dosage. In this case, the state change processor sends a notification to the patient or a caretaker reminding them of the scheduled dosage. In one embodiment, application program interface (API's) are implemented by the medical activity detector and state change processor to establish medical adherence. API's are set of routines, protocols, and tools for building software applications. An API specifies how software components should interact. APIs are used when programming graphical user interface (GU) components. APIs are used by system hardware as well as software applications. For example Get Info API is implemented by the state change processor to invoke the medical activity detector that returns device firmware version along with a recorded event count. Other APIs used are Clear Events API, Get Serial Number API, Get Status API, Set Medicine Info API, Set Temperature Range API, Get Temperature Info API, Get Medicine Info API, and Reset Device API.

The firmware on the device and the app on the smartphone has the capability to filter out the false positives like opening and closing the container too fast, or not opening the container completely. Such false positives if removed from the event queue provide the patient with accurate results.

There are various advantages of the various embodiments described above. The medical device is easy to use and simple to assemble. The medical device is used to send proactive notification to the patient or caretaker. The medical device is used to track the state change such as open and close of the medical device. The medical activity detector includes a waterproof lid to prevent medicine from coming in contact with the medical activity detector which may be avoid corrosion of the medical activity detector and also contamination of the medicine. Patients are proactively reminded of their medicines on time or at the pre-defined time. This ensures that the patients adhere to their prescribed medicines. The medical device ensures complete medical adherence of the patients. The medical device is inexpensive as well,

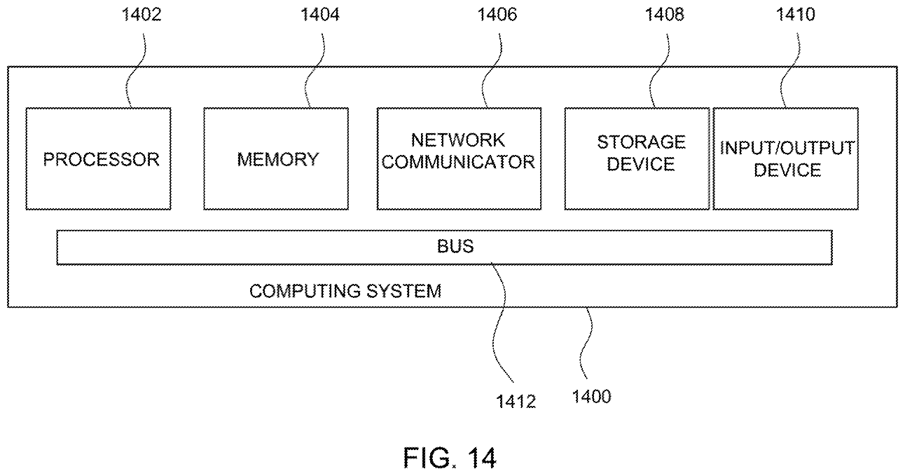

FIG. 14 is a block diagram illustrating a computing system 1400 consistent with implementations of the current subject matter, according to one embodiment. As shown in FIG. 14, the computing system 1400 can include a processor 1402, a memory 1404, network communicator 1406, a storage device 1408, and input/output devices 1410. The processor 1402, the memory 1404, network communicator 1406, the storage device 1408, and the input/output device 1410 can be interconnected via a system bus 1412. The processor 1402 is capable of processing instructions for execution within the computing system 1400. Such executed instructions can implement one or more components of, for example, application A. In some example embodiments, the processor 1402 can be a single-threaded processor. Alternately, the processor 1402 can be a multi-threaded processor. The processor 1402 is capable of processing instructions stored in the memory 1404 and/or on the storage device 1408 to display graphical information for a user interface provided via the input/output device 1410.

The memory 1404 is a computer readable medium such as volatile or non-volatile that stores information within the computing system 1400. The memory 1404 can store instructions and/or other data associated with the processes disclosed herein. The storage device 1408 is capable of providing persistent storage for the computing system 1400. The storage device 1408 can be a hard disk device, an optical disk device, or a tape device, or other suitable persistent storage means. The input/output device 1410 provides input/output operations for the computing system 1400. In some example embodiments, the input/output device 1410 includes a keyboard and/or pointing device. In various implementations, the input/output device 1410 includes a display unit for displaying graphical user interfaces.

According to some example embodiments, the input/output device 1410 can provide input/output operations for a network device. For example, the input/output device 1410 can include Ethernet ports or other networking ports to communicate with one or more wired and/or wireless networks (e.g., a local area network (LAN), a wide area network (WAN), the Internet).

In some example embodiments, the computing system 1400 can be used to execute various interactive computer software applications that can be used for organization, analysis and/or storage of data in various formats. Alternatively, the computing system 1400 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. Upon activation within the applications, the functionalities can be used to generate the user interface provided via the input/output device 1410. The user interface can be generated and presented to a user by the computing system 1400 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitory, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the above description, numerous specific details are set forth to provide a thorough understanding of embodiments. One skilled in the relevant art will recognize, however that the embodiments can be practiced without one or more of the specific details or with other methods, components, techniques, etc. In other instances, well-known operations or structures are not shown or described in detail.

Although the processes illustrated and described herein include series of steps, it will be appreciated that the different embodiments are not limited by the illustrated ordering of steps, as some steps may occur in different orders, some concurrently with other steps apart from that shown and described herein. In addition, not all illustrated steps may be required to implement a methodology in accordance with the one or more embodiments. Moreover, it will be appreciated that the processes may be implemented in association with the apparatus and systems illustrated and described herein as well as in association with other systems not illustrated.

The above descriptions and illustrations of embodiments, including what is described in the Abstract, is not intended to be exhaustive or to limit the one or more embodiments to the precise forms disclosed. While specific embodiments of, and examples for, the one or more embodiments are described herein for illustrative purposes, various equivalent modifications are possible within the scope, as those skilled in the relevant art will recognize. These modifications can be made in light of the above detailed description. Rather, the scope is to be determined by the following claims, which are to be interpreted in accordance with established doctrines of claim construction.

Although the embodiments herein are described with various specific embodiments, it will be obvious for a person skilled in the art to practice the embodiments herein with modifications.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

The scope of the embodiments will be ascertained by the claims to be submitted at the time of filing a complete specification.

What is claimed is:

1. A computer implemented method to auto detect medical adherence, the method comprising:
automatically detecting a state change of a medical device based on a motion of the medical device, wherein the state change is from a first state to a second state;
determining whether a state change processor is within a range of the medical device, wherein the state change processor monitors medical adherence; and
upon determining that the state change processor is within the range of the medical device, sending the state change information to the state change processor.

2. The computer implemented method according to claim 1, further comprising:
automatically placing a medical activity detector in an idle state when the state change is not detected.

3. The computer implemented method according to claim 1, further comprising:
determining the motion when the state change from the first state to the second state is detected, wherein the motion detection is based on the state change of an inertial measurement unit (IMU).

4. The computer implemented method according to claim 2, further comprising:
storing a real time change data when the medical activity detector and the state change detector are within a communication range.

5. The computer implemented method according to claim 2, further comprising:
storing a historical change data when the medical activity detector and the state change detector are outside the communication range when the communication is established.

6. The computer implemented method according to claim 2, further comprising:
sending a notification to the medical activity detector when a state change message is not received at a pre-determined time slot.

7. The computer implemented method according to claim 2, further comprising:
dispense medicine from a medicine container coupled to the medical device, wherein the dispensing of the medicine is via an opening the medical device and the dispensed medicine information is stored in the medical device.

8. A computer system for determining medical adherence comprising:
a computer memory to store program code;
a processor to execute the program code;
a medical device comprising:
a medical activity detector that executes program code to:
automatically detecting a state change of a medical device based on a motion of the medical device, wherein the state change is from a first state to a second state;
determining whether a state change processor is within a range of the medical device, wherein the state change processor monitors medical adherence; and
upon determining that the state change processor is within the range of the medical device, sending the state change information to the state change processor.

9. The computer system of claim 8, further comprising instructions which when executed by the computer cause the computer to:
automatically placing a medical activity detector in an idle state when the state change is not detected; and
automatically waking up the medical activity detector from the idle state when interrupted by the state change.

10. The computer system of claim 8, further comprising instructions which when executed by the computer cause the computer to:
determining the motion when the state change from the first state to the second state is detected, wherein the motion detection is based on the state change of an inertial measurement unit (IMU).

11. The computer system of claim 9, further comprising instructions which when executed by the computer cause the computer to:
storing a real time change data when the medical activity detector and the state change detector are within a communication range.

12. The computer system of claim 9, further comprising instructions which when executed by the computer cause the computer to:
storing a historical change data when the medical activity detector and the state change detector are outside the communication range when the communication is established.

13. The computer system of claim 9, further comprising instructions which when executed by the computer cause the computer to:
sending a notification to the medical activity detector when a state change message is not received at a pre-determined time slot.

14. The computer system of claim 9, further comprising instructions which when executed by the computer cause the computer to:
dispense medicine from a medicine container coupled to the medical device, wherein the dispensing of the medicine is via an opening the medical device and the dispensed medicine information is stored in the medical device.

15. An article of manufacture including a non-transitory computer readable storage medium to tangibly store instructions, which when executed by a computer, cause the computer to:
automatically detecting a state change of a medical device based on a motion of the medical device, wherein the state change is from a first state to a second state;
determining whether a state change processor is within a range of the medical device, wherein the state change processor monitors medical adherence; and
upon determining that the state change processor is within the range of the medical device, sending the state change information to the state change processor.

16. The article of manufacture of claim 15, further comprising instructions which when executed by the computer cause the computer to:
automatically placing a medical activity detector in an idle state when the state change is not detected.

17. The article of manufacture of claim 15, further comprising instructions which when executed by the computer cause the computer to:
determining the motion when the state change from the first state to the second state is detected, wherein the motion detection is based on an inertial measurement unit (IMU).

18. The article of manufacture of claim 16, further comprising instructions which when executed by the computer cause the computer to:
storing a real time change data when the medical activity detector and the state change detector are within a communication range.

19. The article of manufacture of claim 16, further comprising instructions which when executed by the computer cause the computer to:
storing a historical change data when the medical activity detector and the state change detector are outside the communication range when the communication is established.

20. The article of manufacture of claim 16, further comprising instructions which when executed by the computer cause the computer to:
sending a notification to the medical activity detector when a state change message is not received at a pre-determined time slot.
